(19) United States
(12) Patent Application Publication
(10) Pub. No.: US 2022/0039185 A1
(43) Pub. Date: Feb. 3, 2022

(54) RADIATION IMAGING SYSTEM AND CONTROL METHOD OF RADIATION IMAGING SYSTEM

(71) Applicant: CANON KABUSHIKI KAISHA, Tokyo (JP)

(72) Inventors: HIDEKI NONAKA, Kanagawa (JP); NORIHIKO MIYACHI, Tokyo (JP)

(21) Appl. No.: 17/502,609

(22) Filed: Oct. 15, 2021

Related U.S. Application Data
(63) Continuation of application No. PCT/JP2020/016286, filed on Apr. 13, 2020.

(30) Foreign Application Priority Data
Apr. 16, 2019 (JP) 2019-077898
Apr. 16, 2019 (JP) 2019-077933

(51) Int. Cl. H04W 76/15; G01T 7/00; A61B 6/00; H04W 76/11

(52) U.S. Cl. CPC H04W 76/15; H04W 76/11; A61B 6/548; G01T 7/00

(57) ABSTRACT

To establish a wireless connection to a desired radio network by preventing a user from erroneously connecting to another radio network without a special operation in a radiation imaging system. In the radiation imaging system, a radiation imaging apparatus establishes a first wireless connection between the radiation imaging apparatus and a wireless master unit by using first information uniquely indicating itself as an identifier, third information corresponding to the radiation imaging apparatus wirelessly connected to a wireless master unit comprising an access point and a console connected thereto as an identifier, a radiation imaging apparatus uses fourth information according to second information uniquely indicating the wireless master unit itself notified from the wireless master unit through the first wireless connection as an identifier, and the wireless master unit uses fourth information as an identifier to establish a second wireless connection between the radiation imaging apparatus and the wireless master unit.

### CROSS-REFERENCE TO RELATED APPLICATIONS

This application is a Continuation of International Patent Application No. PCT/JP2020/016286, filed Apr. 13, 2020, which claims the benefit of Japanese Patent Application No. 2019-077933, filed Apr. 16, 2019, and the benefit of Japanese Patent Application No. 2019-077898, filed Apr. 16, 2019, all of which are hereby incorporated by reference herein in their entirety.

### BACKGROUND OF THE INVENTION

### Field of the Invention

The present invention relates to a radiation imaging system and a method of controlling the radiation imaging system.

### Description of the Related Art

As an imaging apparatus used for medical image diagnosis and non-destructive inspection by radiation such as X-ray, there is a radiation imaging apparatus using a flat plane detector (Flat Panel Detector, hereinafter abbreviated as FPD) formed of a semiconductor material. In the FPD, a plurality of pixels for converting radiation into electric signals are arranged in a two-dimensional matrix, and a radiation image based on the detected radiation is outputted. Such a radiation imaging apparatus is used, for example, in medical image diagnosis as a digital imaging apparatus for still image photography such as general photography or moving image photography such as fluoroscopic photography.

In recent years, an FPD mounted with a battery as a power source and performing data communication by wireless communication has been used. As a wireless communication system, a wireless LAN (Local Area Network) defined by IEEE 802.11 is widely used. In order to establish a wireless connection between apparatuses in a wireless LAN, it is necessary to match the service set identifier (SSID), authentication method, encryption type, encryption key, and the like between the apparatuses to be connected.

In general, these settings relating to the wireless connection are manually set on all the apparatuses that make the wireless connection, or are set by using a method based on a standardization technology such as WPS (WiFi Protected Setup). In addition, Japanese Patent Application Laid-Open No. 2013-236711 proposes a method in which an RFID or barcode is installed at the entrance of a radiographic chamber and settings necessary for establishing a wireless connection are notified from the RFID or barcode. In addition, Japanese Patent Application Laid-Open No. 2004-234286 proposes a technique for reducing a burden on a user at the time of setting by setting communication setting information for establishing a subsequent wireless connection with a specific communication partner apparatus using communication established by initial communication setting information which does not limit the communication partner apparatus.

However, manual configuration requires human input, and there is a possibility that a wireless connection cannot be established due to an input error. In the PIN code system of the WPS, it is necessary to manually input the same PIN code to the master unit side for the slave unit which outputs the PIN code. In addition, in the push-button system of the WPS, it is necessary to substantially simultaneously press both the buttons of the slave unit and the master unit. According to the technique disclosed in Japanese Patent Application Laid-Open No. 2004-234286, although it is also unnecessary to perform an operation or the like to establish a wireless connection based on initial communication setting information which does not limit a communication partner apparatus, it is necessary to perform an operation by a user to subsequently set communication setting information for establishing a wireless connection with a specific communication partner apparatus. Although it is possible to continue the operation by the wireless connection established by the initial communication setting information, there is a security risk because any apparatus can be connected because the communication partner apparatus is not specified. In addition, the present invention cannot cope with the case where a plurality of wireless network configurations using similar apparatuses are to be operated in a close environment.

### SUMMARY OF THE INVENTION

The present disclosure has been made in view of such circumstances, and it is an object of the disclosure to prevent erroneous connection to other wireless networks and to establish wireless connection to a desired wireless network in a radiation imaging system without any special operation by a user.

Disclosed is a radiation imaging system for wirelessly connecting a control apparatus and a radiation imaging apparatus controlled by the control apparatus via any one of a plurality of access points, comprising:

a first storage unit included within the radiation imaging apparatus, and configured to store first information uniquely indicating the radiation imaging apparatus;

a second storage unit included within at least one of an access point of the plurality of the access point and the control apparatus connected to the access point, and configured to store second information uniquely indicating the access point;

a third storage unit included within at least one of the access point and the control apparatus connected to the access point, and configured to store third information uniquely indicating a radiation imaging apparatus that can be wirelessly connected to the access point;

a first control unit included within the radiation imaging apparatus, configured to establish a first wireless connection with a first access point of the plurality of access points by using the first information as an identifier, and receive fourth information corresponding to the second information through the first wireless connection so as to establish a second wireless connection with the first access point by using the fourth information as the identifier; and

a second control unit included within the access point, configured to establish the first wireless connection with the radiation imaging apparatus using the third information corresponding to the selected radiation imaging apparatus as an identifier, so as to transmit the fourth information through the first wireless connection, and establish the second wireless connection with the radiation imaging apparatus using the fourth information as the identifier.

In addition, disclosed is a radiation imaging system for wirelessly connecting a control apparatus and a radiation imaging apparatus controlled by the control apparatus via any one of a plurality of access points, comprising:

a first storage unit included within the radiation imaging apparatus, and configured to store first information uniquely indicating the radiation imaging apparatus;

a second storage unit included within at least one of an access point of the plurality of the access points and the control apparatus connected to the access point, and configured to store second information uniquely indicating the access point;

a first control unit included within the radiation imaging apparatus, and configured to establish a first wireless connection that does not specify a communication partner, transmit the first information through the first wireless connection, receive third information corresponding to the second information through the first wireless connection, and establish a second wireless connection with an access point of the plurality of access points by using the third information as an identifier; and

a second control unit included within the access point, and configured to establish the first wireless connection that does not specify a communication partner, receive the first information through the first wireless connection, transmit the third information to establish the second wireless connection with the radiation imaging apparatus using the third information as an identifier.

Further features of the present invention will become apparent from the following description of exemplary embodiments with reference to the attached drawings.

### BRIEF DESCRIPTION OF THE DRAWINGS

FIG. 1 is a diagram showing a configuration example of a radiation imaging system according to the first embodiment.

FIG. 2 is a diagram showing a configuration example of a radiation imaging apparatus according to the first embodiment.

FIG. 3 is a diagram showing a configuration example of a wireless master unit according to the first embodiment.

FIG. 4 is a flowchart showing an operation example of the radiation imaging system (wireless slave side) in the first embodiment.

FIG. 5 is a flowchart showing an operation example of a radiation imaging system (wireless master unit side) in the first embodiment.

FIG. 6 illustrates the operation of the radiation imaging system of the first embodiment.

FIG. 7 is a diagram showing a configuration example of a wireless master unit according to the second embodiment.

FIG. 8 is a flowchart showing an operation example of the radiation imaging system (wireless slave side) in the second embodiment.

FIG. 9 is a flowchart showing an operation example of a radiation imaging system (wireless master unit side) in the second embodiment.

FIG. 10 is a diagram illustrating the operation of the radiation imaging system according to the second embodiment.

FIG. 11 is a diagram showing a configuration example of a wireless master unit according to the third embodiment.

FIG. 12 is a flowchart showing an operation example of the radiation imaging system (wireless slave side) in the third embodiment.

FIG. 13 is a flowchart showing an operation example of a radiation imaging system (wireless master unit side) in the third embodiment.

### DESCRIPTION OF THE EMBODIMENTS

Embodiments of the present disclosure will be described below with reference to the drawings. It should be noted that the embodiments described below are not intended to limit the claimed invention, and not all combinations of features described in the embodiments are essential as means for the present disclosure. The radiation in the present embodiment may include, in addition to a-rays, p-rays, y-rays, etc., which are beams produced by particles (including photons) emitted by radiation decay, beams having energy of the same or higher, for example, X-rays, particle rays, cosmic rays, etc.

### First Embodiment

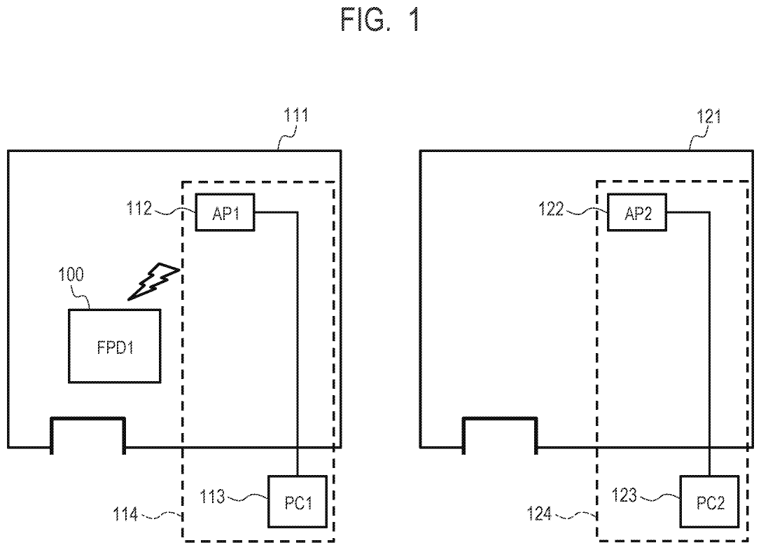

A first embodiment of the present disclosure will be described. FIG. 1 is a diagram showing an example of a radiation imaging system according to the first embodiment of the present disclosure. The radiation imaging system according to the present embodiment has a first radiation imaging chamber 111 and a second radiation imaging chamber 121, and uses a radiation imaging apparatus (FPD1) 100 as a wireless slave unit by moving it to the first radiation imaging chamber 111 and the second radiation imaging chamber 121.

A first access point (AP1) 112 and a first console (PC1) 113 are installed in the first radiation imaging chamber 111. A second access point (AP2) 122 and a second console (PC2) 123 are installed in the second radiation imaging chamber 121. In this embodiment, for example, the first access point 112 and the first console 113 constitute the first wireless master unit 114. For example, the second access point 122 and the second console 123 constitute the second wireless master unit 124.

In the radiation imaging system of this embodiment, the identifiers of the first access point 112 and the second access point 122 in the initial state are not set. Therefore, in the initial state, the radiation imaging apparatus 100 serving as the wireless slave unit is not connected to both the first access point 112 and the second access point 122. In this embodiment, an SSID (Service Set Identifier) will be described as an identifier, but the present invention is not limited thereto. For example, an encryption key used for authentication at the time of connection may be applied as the identifier, or both the SSID and the encryption key may be applied.

Although FIG. 1 shows a case where there are two sets of an access point and a console and one radiation imaging apparatus, the radiation imaging system in this embodiment is not limited thereto. The number of access point and console pairs and the number of radiation imaging apparatuses in a radiation imaging system are each arbitrary.

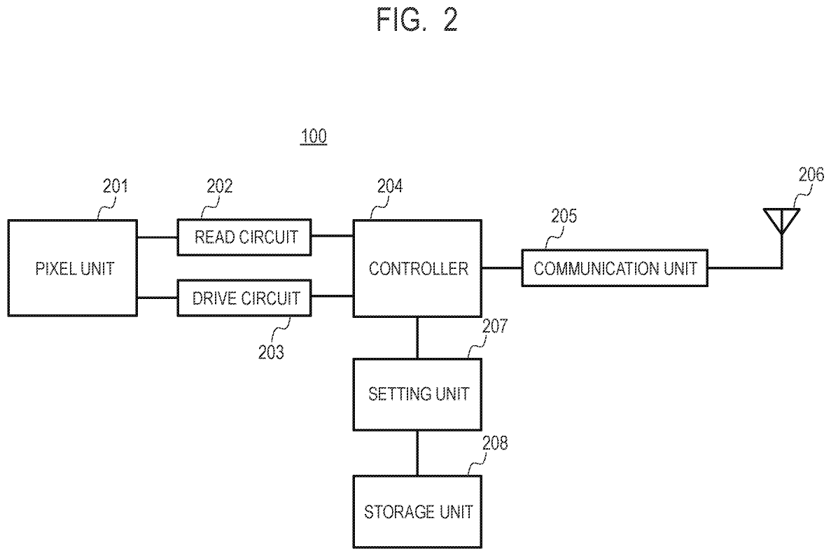

FIG. 2 is a block diagram showing a configuration example of the radiation imaging apparatus 100 according to the present disclosure. The radiation imaging apparatus 100 is a radiation imaging apparatus using, for example, a flat panel detector (FPD) formed of a semiconductor material. The radiation imaging apparatus 100 includes a pixel unit 201, a read circuit 202, a drive circuit 203, a controller 204, a communication unit 205, a communication interface 206, a setting unit 207, and a storage unit 208.

The pixel unit 201 has a plurality of pixels arranged in a two-dimensional matrix (two-dimensional matrix). Each of the plurality of pixels has a conversion element for converting incident radiation into an electric charge and accumulating the electric charge, and a switching element for transferring the accumulated electric charge. The conversion element may be an element for directly converting radiation into charge, or may be constituted by combining a scintillator for converting radiation into visible light and an element for converting visible light into charge. The switching element is composed of a TFT (Thin Film Transistor), for example, and is on/off controlled by a signal input to a gate terminal.

The gate terminal of the switching element of each pixel in the same row of the pixel unit 201 is connected to one gate signal line extending in the row direction. A plurality of gate signal lines are arranged in the column direction and connected to the drive circuit 203. The output terminals of the switching elements arranged in the same column are connected to one data signal line extending in the column direction. Like the gate signal lines, a plurality of data signal lines are arranged in the row direction and connected to the read circuit 202. That is, the switching elements arranged on the same row are simultaneously turned on/off, and their outputs are output to the read circuit 202 in a row unit.

The read circuit 202 samples and holds the charge inputted through the data signal line after voltage conversion, amplifies it by an amplifier, and then analog-to-digital conversion is performed. In this manner, the read circuit 202 acquires the amount of charge accumulated in each pixel of the pixel unit 201 as a digital value. A drive circuit 203 drives a gate signal line. A drive circuit 203 and a read circuit 202 cooperate with each other to perform reading operation by turning on the switching elements of the same row by a gate signal for all rows while switching the rows in order to acquire digital data for one image.

The controller 204 is constituted by, for example, a CPU (Central Processing Unit) or the like, and controls each functional part of the radiation imaging apparatus 100. The controller 204 performs control related to, for example, the read circuit 202 and the drive circuit 203 for acquiring a radiation image based on the radiation detected by the pixel unit 201. The controller 204 performs control for establishing a wireless connection based on an identifier set by the setting unit 207 for performing wireless communication using the communication unit 205 and the communication interface 206, for example.

The communication unit 205 transmits and receives a radio signal to and from a partner apparatus via a communication interface 206 such as an antenna, for example. The setting unit 207 sets information relating to the wireless connection as an identifier in the controller 204. The storage unit 208 stores various data including information related to wireless connection. The storage unit 208 previously stores first information uniquely indicating the radiation imaging apparatus 100 itself in a communication network.

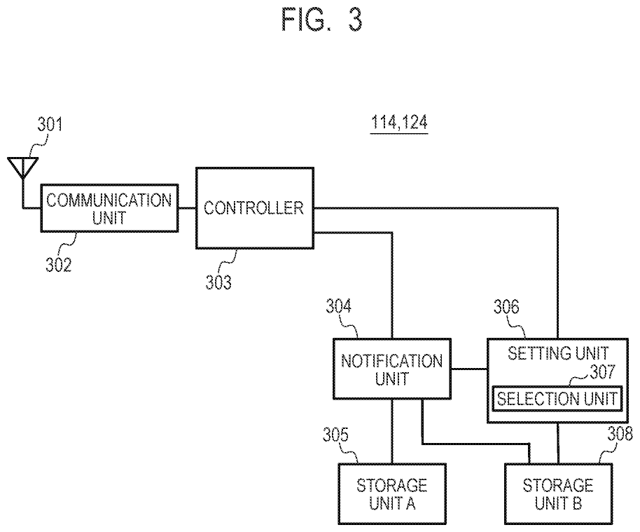

FIG. 3 is a block diagram showing a configuration example of the wireless master units 114 and 124 in the present embodiment. Each of the wireless master units 114 and 124 respectively includes a communication interface 301, a communication unit 302, a controller 303, a notification unit 304, a storage unit A 305, a setting unit 306, a selection unit 307, and a storage unit B 308. In the present embodiment, for example, each of the access points 112 and 122 respectively has a communication interface 301, a communication unit 302, and a controller 303, and each of the consoles 113 and 123 respectively has a notification unit 304, a storage unit A 305, a setting unit 306, a selection unit 307, and a storage unit B 308. It should be noted that the access points 112 and 122 may have some or all of the functional parts of the consoles 113 and 123, and control instructions or the like may be given from the consoles 113 and 123 as necessary to realize the functions of the wireless master units 114 and 124.

The communication unit 302 transmits and receives a radio signal to and from a partner device via a communication interface 301 such as an antenna, for example. The controller 303 includes, for example, a CPU (Central Processing Unit) and performs various kinds of control. The controller 303 performs control for establishing a wireless connection based on an identifier set by the setting unit 306 for performing wireless communication using the communication interface 301 and the communication unit 302, for example.

A notification unit 304 notifies information related to wireless connection to a radiation imaging apparatus 100 being a communication partner apparatus via a controller 303. The notification unit 304 outputs to the setting unit 306 information relating to the wireless connection to be notified to the radiation imaging apparatus 100. The notification unit 304 generates the fourth information relating to the wireless connection based on, for example, the second information acquired from the storage unit A 305 and the third information acquired from the storage unit B 308, and notifies the radiation imaging apparatus 100 of the generated fourth information and outputs it to the setting unit 306. The storage unit A 305 stores various data including information related to wireless connection. In the storage unit A 305, second information uniquely indicating the wireless master units 114 and 124 themselves in the communication network is previously stored.

The setting unit 306 sets information relating to the wireless connection as an identifier in the controller 303. The selection unit 307 selects information related to the wireless connection corresponding to the radiation imaging apparatus 100 to be connected from information related to the wireless connection stored in the storage unit B 308. The storage unit B 308 stores various data including information related to wireless connection. In the storage unit B 308, one or more third information that uniquely indicate each of the wireless slave units assumed to be wirelessly connected to the wireless master units 114 and 124 in the communication network are previously stored. That is, in the storage unit B 308, third information that uniquely indicates each radiation imaging apparatus 100 in the communication network is stored in advance for each radiation imaging apparatus 100 that can be wirelessly connected to the wireless master units 114 and 124.

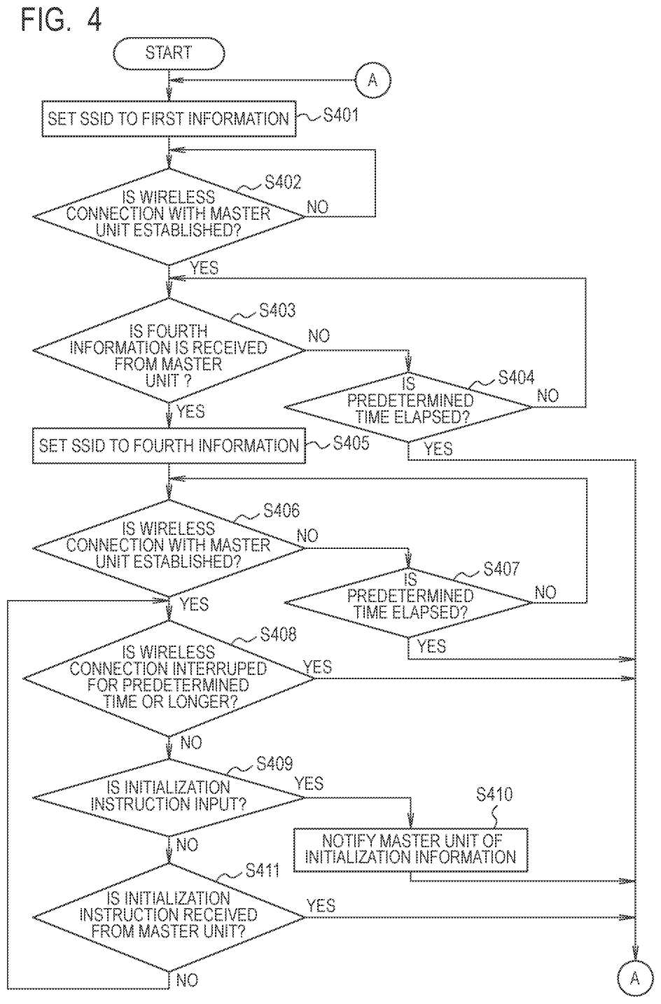

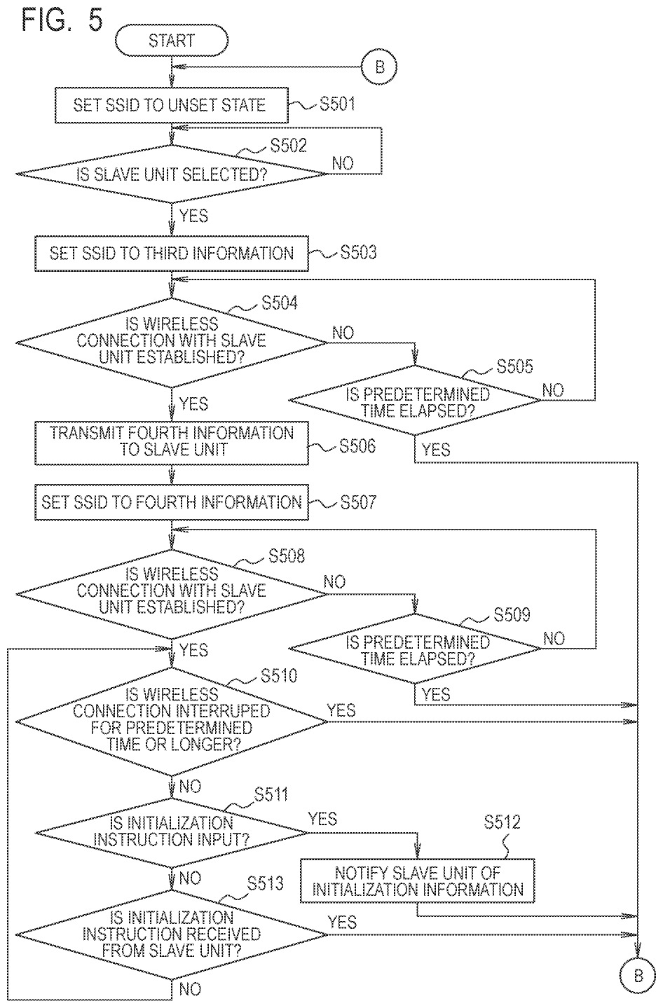

Next, with reference to FIGS. 4 and 5, an operation related to wireless connection in the radiation imaging system of this embodiment will be described. FIG. 4 is a flowchart showing an operation example related to wireless connection in the radiation imaging system (wireless slave unit side) in this embodiment, and FIG. 5 is a flowchart showing an operation example related to wireless connection in the radiation imaging system (wireless master unit side) in this embodiment. Hereinafter, a case where a wireless connection is established between the radiation imaging apparatus 100 and the first wireless master unit 114 constituted by the first access point 112 and the first console 113 will be described by way of example as appropriate.

In a communication network, first information uniquely indicating a radiation imaging apparatus 100 is referred to as “FPD1”, and second information uniquely indicating a first wireless master unit 114 is referred to as “AP1”. The wireless connection based on the information (“FPD1” indicates the radiation imaging apparatus 100 in the following example) uniquely indicating the radiation imaging apparatus is referred to as a “first wireless connection”. The wireless connection based on the information (“FPD1-AP1” indicates a combination of the radiation imaging apparatus 100 and the first wireless master unit 114 in the following example.) uniquely indicating the combination of the radiation imaging apparatus and the wireless master unit is referred to as a “second wireless connection”.

First, an operation related to wireless connection in the radiation imaging apparatus 100, which is a wireless slave unit side shown in FIG. 4, will be described. In a radiation imaging system, communication setting is initialized in each apparatus immediately after power is supplied to each apparatus. In the radiation imaging apparatus 100, in step S401, the setting unit 207 reads out the first information uniquely indicating the radiation imaging apparatus 100 itself stored in the storage unit 208, and sets the controller 204 as an identifier for establishing a wireless connection with the wireless master unit. In this example, the setting unit 207 sets the SSID that is the identifier of the controller 204 to “FPD1”. As a result, the radiation imaging apparatus 100 can be connected to the wireless master unit in which SSID is set to “FPD1”.

Next, in step S402, the controller 204 determines whether or not a first wireless connection with the wireless master unit has been established. The controller 204 repeats the processing of step S402 until it is determined that the first wireless connection with the wireless master unit is established, and when it is determined that the first wireless connection with the wireless master unit is established, the process proceeds to step S403. In this example, in the first wireless master unit 114, the same “FPD1” as the first information indicating the radiation imaging apparatus 100 is set as the SSID, whereby the first wireless connection can be established, and in this state, the wireless connection can be established to enable mutual communication.

In step S403 after the first wireless connection is established, the controller 204 determines whether or not the fourth information used as the SSID in the second wireless connection is received from the wireless master unit side through the first wireless connection. If the controller 204 determines that the fourth information has not been received from the wireless master unit (NO), the process proceeds to step S404, and if the controller 204 determines that the fourth information has been received from the wireless master unit (YES), the process proceeds to step S405.

Here, the fourth information is information uniquely indicating a set of the radiation imaging apparatus and the wireless master unit to be wirelessly connected, and is preferably information generated, for example, based on the first information uniquely indicating the radiation imaging apparatus and the second information uniquely indicating the wireless master unit. In this example, “FPD1-AP1”, which is designated by connecting the information “FPD1” indicating the radiation imaging apparatus 100 and the information “AP1” indicating the first wireless master unit 114 with a hyphen “-”, is defined as the fourth information. The fourth information generation method is not limited to this, and encryption may be performed by using an exclusive OR of both character strings, and the generation algorithm thereof is not limited. Further, the current SSID “FPD1” is information recognized by both the radiation imaging apparatus 100 and the first wireless master unit 114. Therefore, a method may be employed in which only the information “AP1” indicating the first wireless master unit 114 is transmitted from the first wireless master unit 114 as the fourth information, and the SSID of the second wireless connection is generated by the radiation imaging apparatus 100 together with the current SSID of “FPD1”. In this case, it is necessary to use the same generation algorithm in the radiation imaging apparatus 100 and the first wireless master unit 114.

In step S404, the controller 204 determines whether or not a predetermined period of time has elapsed since it determined that the first wireless connection has been established. If the controller 204 determines that the predetermined time has not elapsed (NO), the process returns to step S403. On the other hand, if the controller 204 determines that the predetermined time has elapsed (YES), the process returns to step S401 to initialize the SSID. At this point in time, since the SSID is not updated from the first information set in step S401, the return destination of the process in the case where the controller 204 determines that the predetermined time has elapsed (YES) may be set to step S402.

In step S405, the setting unit 207 sets the acquired fourth information to the controller 204 as an identifier for establishing a second wireless connection with the wireless master unit. In this example, the setting unit 207 sets the SSID of the controller 204 to “FPD1-AP1”.

Next, in step S406, the controller 204 determines whether or not a second wireless connection with the wireless master unit has been established. If the controller 204 determines that the second wireless connection with the wireless master unit has not been established (NO), the process proceeds to step S407, and if the controller 204 determines that the second wireless connection with the wireless master unit has been established (YES), the process proceeds to step S408. In this example, when the fourth information “FPD1-AP1” is set as the SSID in the first wireless master unit 114, the second wireless connection can be established, and in this state, the wireless connection can be established to enable mutual communication.

In step S407, the controller 204 determines whether or not a predetermined period of time has elapsed since it determined that the fourth information has been received. If the controller 204 determines that the predetermined time has not elapsed (NO), the process returns to step S406. On the other hand, if the controller 204 determines that the predetermined time has elapsed (YES), the process returns to step S401 to initialize the SSID.

When the controller 204 determines in step S406 that the second wireless connection with the wireless master unit has been established, thereafter, data communication and the like are performed between the radiation imaging apparatus and the wireless master unit through the second wireless connection based on the fourth information uniquely indicating the combination. For example, in a state where the second wireless connection is maintained, photographing using the radiation imaging apparatus 100 is performed by the control software on the console. At this time, a command for controlling the radiation imaging apparatus 100, data indicating the state of the radiation imaging apparatus 100, radiation image data acquired by photographing, and the like are transmitted and received by the second wireless connection.

Here, it is assumed that, after the necessary photographing is performed, for example, the radiation imaging apparatus 100 is taken out for use in another radiation imaging room. In this case, since the identifier with the wireless master unit corresponding to the radiation imaging room used so far remains, the radiation imaging apparatus 100 cannot wirelessly connect with the wireless master unit corresponding to another radiation imaging room. It is necessary to initialize the current identifier in order to enable wireless connection with a wireless master unit corresponding to another radiographic room.

For example, when the second wireless connection based on the fourth information is interrupted for a predetermined time or more, it can be considered that the radiation imaging apparatus 100 has been taken out to another radiation imaging room. Therefore, as shown in step S408, the controller 204 determines whether or not the second wireless connection has been interrupted for a predetermined period of time or longer. If the controller 204 determines that the second wireless connection has been interrupted for a predetermined time or longer (YES), the process returns to step S401 to initialize the SSID.

Further, for example, the user may be able to input the initialization of the SSID via an operation unit (not shown) of the radiation imaging apparatus 100, and as shown in step S409, the controller 204 determines whether or not the initialization of the SSID has been input. If it is determined that the SSID has been initialized (YES), the controller 204 notifies the wireless master unit of the SSID initialization instruction in step S410, and then process returns to step S401 to initialize the SSID.

Further, for example, it is also possible to allow a user to input the initialization of the SSID from a touch panel or the like of a console constituting the wireless master unit, and in the wireless master unit, when there is the initialization input of the SSID, the wireless slave unit may be notified of the initialization instruction of the SSID. In this case, as shown in step S411, the controller 204 determines whether or not an SSID initialization instruction has been received from the wireless master unit. If the controller 204 determines that an SSID initialization instruction has been received from the wireless master unit (YES), the process returns to step S401 to initialize the SSID.

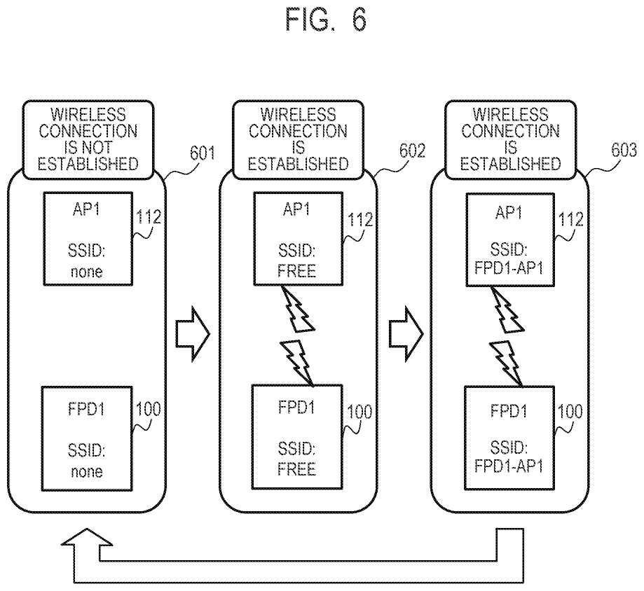

Next, an operation shown in FIG. 5 related to wireless connection on the wireless master unit side will be described. In the radiation imaging system, communication setting is initialized in each apparatus immediately after power is supplied to each apparatus. In the wireless master units 114 and 124, in step S501, the setting unit 306 sets the identifier of the controller 303 to an unset state. As a result, the wireless master units 114 and 124 do not establish wireless connection with any of the radiation imaging apparatuses. At this time, the states of the first access point 112 and the radiation imaging apparatus 100 in this embodiment become the state 601 shown in FIG. 6. It is assumed that the processing of step S401 shown in FIG. 4 has been completed in the radiation imaging apparatus 100.

Although the identifier is not set here, the second information (In this example, the information “AP1” indicating the first wireless master unit 114 is used.) uniquely indicating the wireless master units 114 and 124 themselves stored in the storage unit A 305 may be set as the identifier. In this case, the presence of the wireless master units 114 and 124 can be known from the radiation imaging apparatus 100 by the SSID broadcast.

Next, in step S502, the setting unit 306 determines whether or not a radiation imaging apparatus (wireless slave unit to be wirelessly connected) to be used for photographing is selected at the console of the wireless master unit. The radiation imaging apparatus is selected by, for example, displaying a list of radiation imaging apparatuses stored in the storage unit B 308 on a screen of a console, and a user selects a desired radiation imaging apparatus by using a touch panel or the like. If the setting unit 306 determines that the radiation imaging apparatus used for photographing is selected, the process proceeds to step S503, and if not, the process waits until the radiation imaging apparatus used for photographing is selected.

In step S503, the setting unit 306 reads out the third information corresponding to the selected radiation imaging apparatus 100 to be used for photographing from among the third information previously stored in the storage unit B 308. Then, the setting unit 306 sets the read third information in the controller 303 as an identifier for establishing a wireless connection with the radiation imaging apparatus 100 used for photographing. In the storage unit B 308, third information that uniquely indicates each of a plurality of radiation imaging apparatuses assumed to be used for photographing is previously stored. That is, the third information is the same information as the first information stored in the storage unit 208 of each radiation imaging apparatus. In this example, the setting unit 306 sets the SSID that is the identifier of the controller 303 to “FPD1” that is the third information corresponding to the radiation imaging apparatus 100.

Here, as a method for storing the third information in the storage unit B 308, the user or the installer may perform the registration operation manually at the time of installation of the radiation imaging system. Further, a function of connecting the radiation imaging apparatus and the wireless master unit by a communication unit (not shown) other than the wireless communication shown here and copying the first information stored in the storage unit 208 of the radiation imaging apparatus to the storage unit 308 may be additionally provided.

Next, in step S504, the controller 303 determines whether or not a first wireless connection with the radiation imaging apparatus 100 has been established. If the controller 303 determines that the first wireless connection with the radiation imaging apparatus 100 is not established (NO), the process proceeds to step S505. On the other hand, if the controller 303 determines that the first wireless connection with the radiation imaging apparatus 100 has been established (YES), the process proceeds to step S506. In this example, when “FPD1” is set as the SSID in the radiation imaging apparatus 100, the first wireless connection can be established, and the wireless connection can be established in this state to enable mutual communication.

In step S505, the controller 303 determines whether or not a predetermined period of time has elapsed since the SSID was set to the third information. If the controller 303 determines that the predetermined time has not elapsed (NO), the process returns to step S504. On the other hand, if the controller 303 determines that the predetermined of time has elapsed (YES), the process returns to step S501 to initialize the SSID.

When the first wireless connection is established, the states of the first access point 112 and the radiation imaging apparatus 100 in the present embodiment become the state 602 shown in FIG. 6. In step S506 after the first wireless connection is established, the notification unit 304 generates fourth information for use as an SSID in the second wireless connection, and transmits the fourth information to the radiation imaging apparatus 100 on the wireless slave unit side via the controller 303. The notification unit 304 outputs the generated fourth information to the setting unit 306.

Next, in step S507, the setting unit 306 sets the fourth information generated in step S506 in the controller 303 as an identifier for establishing a second wireless connection with the radiation imaging apparatus 100. In this example, the setting unit 306 sets the SSID of the controller 303 to “FPD1-AP1”.

Next, in step 508, the controller 303 determines whether or not a second wireless connection with the radiation imaging apparatus 100 has been established. If the controller 303 determines that the second wireless connection with the radiation imaging apparatus 100 is not established (NO), the process proceeds to step S509. On the other hand, when the controller 303 determines that the second wireless connection with the radiation imaging apparatus 100 has been established (YES), the process proceeds to step S510. In this example, when the fourth information “FPD1-AP1” is set as the SSID in the first wireless master unit 114, the second wireless connection can be established, and in this state, the wireless connection can be established to enable mutual communication.

In step S509, the controller 303 determines whether or not a predetermined period of time has elapsed since the SSID was set to the fourth information. If the controller 303 determines that the predetermined time has not elapsed (NO), the process returns to step S508. On the other hand, if the controller 303 determines that the predetermined time has elapsed (YES), the process returns to step S501 to initialize the SSID.

When the second wireless connection is established, the states of the first access point 112 and the radiation imaging apparatus 100 in this example become the state 603 shown in FIG. 6. In state 603, since the first access point 112 and the radiation imaging apparatus 100 implement unique wireless connection based on the SSID that uniquely indicates the combination of both, erroneous connection to other wireless networks is prevented.

In Step S508, when the controller 303 determines that a second wireless connection with the radiation imaging apparatus 100 has been established, thereafter, data communication and the like are performed between the radiation imaging apparatus 100 and the wireless master unit through a second wireless connection based on fourth information uniquely indicating the combination. For example, in a state where the second wireless connection is maintained, photographing using the radiation imaging apparatus 100 is performed by the control software on the console, and various commands, various data, and the like are transmitted and received by the second wireless connection.

Here, it is assumed that after the necessary photographing is performed, for example, the radiation imaging apparatus 100 is taken out for use in another radiation imaging room. In this case, when another radiation imaging apparatus is brought in, wireless connection with the radiation imaging apparatus becomes impossible if the previous identifier remains. To allow wireless connection to another radiation imaging apparatus, the current identifier must be initialized.

For example, when the second wireless connection based on the fourth information is interrupted for a predetermined time or more, it can be considered that the radiation imaging apparatus 100 has been taken out to another radiation imaging room. Therefore, as shown in step S510, the controller 303 determines whether or not the second wireless connection has been interrupted for a predetermined period of time or longer. If the controller 303 determines that the second wireless connection has been interrupted for a predetermined time or longer (YES), the process returns to step S501 to initialize the SSID.

Further, for example, the user may be able to input the initialization of the SSID via the console, and as shown in step S511, the controller 303 determines whether or not the initialization of the SSID has been input. If it is determined that the SSID has been initialized (YES), the controller 303 notifies the wireless slave unit side of the SSID initialization instruction in step S512, and then returns to step S501 to initialize the SSID.

Further, for example, it is also possible to enable a user to input the initialization of the SSID via an operation unit (not shown) of the radiation imaging apparatus, and in the radiation imaging apparatus, when there is the initialization input of the SSID, the initialization instruction of the SSID may be notified to the wireless master unit side. In this case, as shown in step S513, the controller 303 determines whether or not an SSID initialization instruction has been received from the wireless slave unit side. If the controller 303 determines that an SSID initialization instruction has been received from the wireless slave unit side (YES), the process returns to step S501 to initialize the SSID.

According to the present embodiment, a first wireless connection between the radiation imaging apparatus and the wireless master unit is established based on information that uniquely indicates the radiation imaging apparatus. A second wireless connection between the radiation imaging apparatus and the wireless master unit is established based on fourth information uniquely indicating the combination of the radiation imaging apparatus and the wireless master unit notified through the first wireless connection. Thus, the wireless connection to the desired wireless network can be established without any special operation by the user.

Here, it is assumed that in a state where the first wireless connection between the radiation imaging apparatus 100 and the first wireless master unit 114 is established, for example, the radiation imaging apparatus 100 is selected by the second wireless master unit 124, and the processes of steps S501 to S503 shown in FIG. 5 are executed. In this case, also in the second wireless master unit 124, a situation occurs in which “FPD1” indicating the radiation imaging apparatus 100 is set as the SSID of the controller 303, and the disadvantageous in which the connection destination of the radiation imaging apparatus 100 cannot be specified (connection to an unintended wireless network) may occur.

On the other hand, in the present embodiment, when the first wireless connection between the radiation imaging apparatus and the wireless master unit is established, the identifier is changed to fourth information uniquely indicating the combination of the radiation imaging apparatus and the wireless master unit to establish a second wireless connection between the radiation imaging apparatus and the wireless master unit. As described above, since the combination of the radiation imaging apparatus and the wireless master unit is wirelessly connected using information that uniquely indicates the combination, it is possible to prevent an unintentional misconnection to another wireless network. Further, by performing the initialization of the SSID in the case where the radiation imaging apparatus of the connection destination cannot be found (the wireless connection cannot be established) within the predetermined time by the processing of step S505 shown in FIG. 5, the possibility of the occurrence of the erroneous connection to the other wireless network can be eliminated as much as possible. In order to prevent erroneous connection to other wireless networks, it is desirable to adjust the radio intensity so that the communicable range of the wireless master units 114 and 124 is limited to the corresponding radiation imaging chambers 111 and 121.

### Second Embodiment

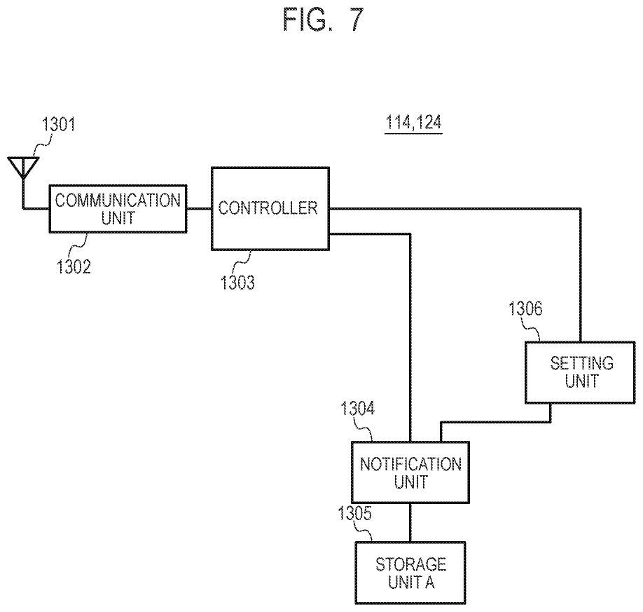

FIG. 7 is a block diagram showing a configuration example of the wireless master units 114 and 124 in the second embodiment. It should be noted that the radiation imaging system of the present embodiment is similar in main configuration to the radiation imaging system of the first embodiment shown in FIG. 1. The radiation imaging system according to the present embodiment includes a first radiation imaging chamber 111 and a second radiation imaging chamber 121. A first access point (AP1) 112 and a first console (PC1) 113 are installed in a first radiation imaging chamber 111 to constitute a first wireless master unit 114. A second access point (AP2) 122 and a second console (PC2) 123 are installed in the second radiation imaging chamber 121 to constitute a second wireless master unit 124. Similarly to the radiation imaging apparatus 100 of the first embodiment, the radiation imaging apparatus as a wireless slave unit also includes a pixel unit 201, a read circuit 202, a drive circuit 203, a controller 204, a communication unit 205, a communication interface 206, a setting unit 207, and a storage unit 208. Therefore, these reference numbers will be used for the configuration of the wireless slave unit in the following description.

Each of the wireless master units 114 and 124 in this embodiment respectively has a communication interface 1301, a communication unit 1302, a controller 1303, a notification unit 1304, a storage unit A 1305, and a setting unit 1306. In the present embodiment, for example, each of the access points 112 and 122 respectively has a communication interface 1301, a communication unit 1302, and a controller 1303, and each of the consoles 113 and 123 has a notification unit 1304, a storage unit A 1305, and a setting unit 1306. It should be noted that the access points 112 and 122 may have some or all of the functional parts of the consoles 113 and 123, and control instructions or the like may be given from the consoles 113 and 123 as necessary to realize the functions of the wireless master units 114 and 124.

A notification unit 1304 notifies information related to wireless connection to a radiation imaging apparatus 100 being a communication partner apparatus via a controller 1303. The notification unit 1304 outputs to the setting unit 1306 information relating to the wireless connection to be notified to the radiation imaging apparatus 100. The notification unit 1304 generates the third information relating to the wireless connection based on, for example, the first information received from the radiation imaging apparatus 100 and the second information acquired from the storage unit A 1305, and notifies the radiation imaging apparatus 100 of the generated third information and outputs it to the setting unit 1306. The storage unit A 1305 stores various data including information related to wireless connection. In the storage unit A 1305, second information uniquely indicating the wireless master units 114 and 124 themselves in the communication network is previously stored. The setting unit 1306 sets information relating to the wireless connection including the identifier in the controller 1303.

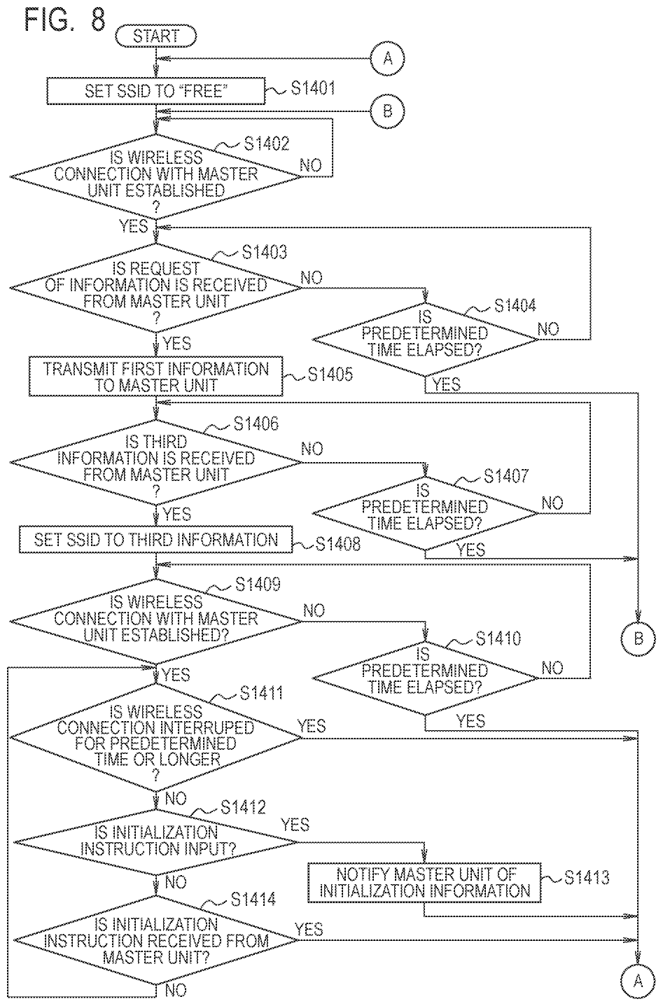

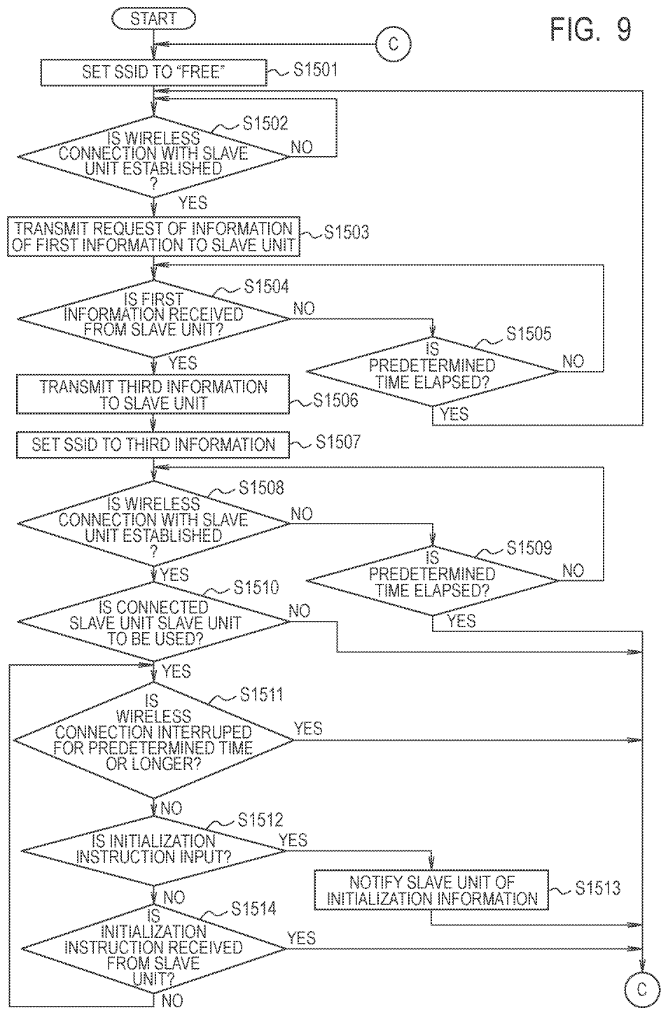

Next, referring to FIGS. 8 and 9, an operation related to wireless connection in the radiation imaging system of the second embodiment will be described. FIG. 8 is a flowchart showing an operation example related to wireless connection in the radiation imaging system (wireless slave unit side) in the second embodiment, and FIG. 9 is a flowchart showing an operation example related to wireless connection in the radiation imaging system (wireless master unit side) in the second embodiment. Hereinafter, a case where a wireless connection is established between the radiation imaging apparatus 100 and the first wireless master unit 114 constituted by the first access point 112 and the first console 113 will be described by way of example as appropriate.

In a communication network, first information uniquely indicating a radiation imaging apparatus 100 is referred to as “FPD1” and second information uniquely indicating a first wireless master unit 114 is referred to as “AP1”. A wireless connection based on information that does not specify a communication partner (“FREE” in the following example) is referred to as a “first wireless connection”. The wireless connection based on the information (In the following example, “FPD1-AP1” indicates a combination of the radiation imaging apparatus 100 and the first wireless master unit 114.) uniquely indicating the combination of the radiation imaging apparatus and the wireless master unit is referred to as a “second wireless connection”.

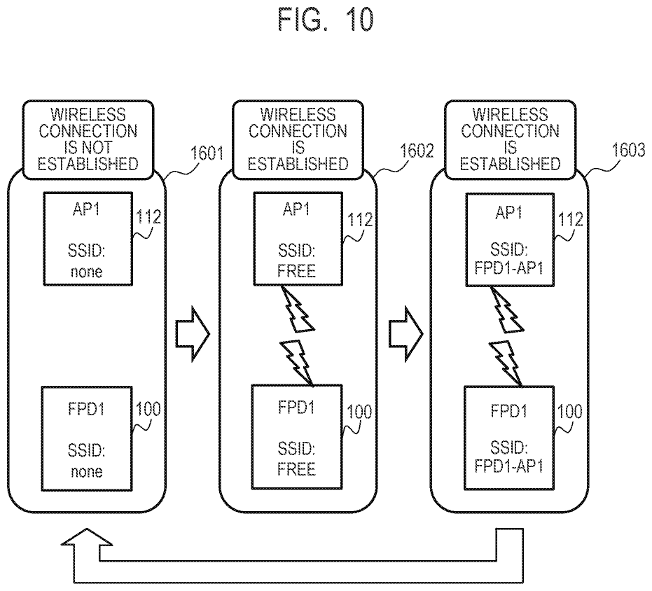

First, an operation related to wireless connection in the radiation imaging apparatus 100, which is a wireless slave unit side shown in FIG. 8, will be described. In the radiation imaging system, in the initial state 1601 shown in FIG. 10, the wireless connection is not established because the wireless connection is not set. In the radiation imaging system, communication settings are initialized in each of the radiation imaging apparatus 100 and the access point 112 immediately after the system is started upon power-on. In the radiation imaging apparatus 100, in step S1401, the setting unit 207 sets an identifier for establishing a first wireless connection that does not specify a communication partner to the controller 204. In this example, the setting unit 207 sets the SSID that is the identifier of the controller 204 to “FREE”. As a result, as shown in the state 1601 of FIG. 10, the radiation imaging apparatus 100 can establish a wireless connection that does not specify a communication partner.

Next, in step S1402, the controller 204 determines whether or not a first wireless connection with the wireless master unit has been established. The controller 204 repeats the processing of step S1402 until it is determined that the first wireless connection with the wireless master unit is established, and when it is determined that the first wireless connection with the wireless master unit is established, the process proceeds to step S1403. In this example, the information “FREE” that does not specify the communication partner is set as the SSID in the first wireless master unit 114, so that the first wireless connection can be established, and in this state, the wireless connection can be established to communicate with each other.

In step S1403 after the first wireless connection is established, the controller 204 determines whether or not the information request of the first information uniquely indicating the radiation imaging apparatus 100 itself has been received from the wireless master unit side through the first wireless connection. If the controller 204 determines that the information request of the first information has not been received from the wireless master unit (NO), the process proceeds to step S1404, and if the controller 204 determines that the information request of the first information has been received from the wireless master unit (YES), the process proceeds to step S1405.

In step S1404, the controller 204 determines whether or not a predetermined period of time has elapsed since it determined that the first wireless connection has been established. If the controller 204 determines that the predetermined time has not elapsed (NO), the process returns to step S1403. On the other hand, when the controller 204 determines that the predetermined time has elapsed (YES), the process returns to step S1402.

In step S1405, the controller 204 acquires first information uniquely indicating the radiation imaging apparatus 100 itself, which is stored in the storage unit 208, and transmits the first information to the wireless master unit via the first wireless connection.

In step S1406 after transmitting the first information, the controller 204 determines whether or not the third information for use as the SSID in the second wireless connection has been received from the wireless master unit side through the first wireless connection. If the controller 204 determines that the third information has not been received from the wireless master unit (NO), the process proceeds to step S1407, and if controller 204 determines that the third information has been received from the wireless master unit (YES), the process proceeds to step S1408.

The third information is information uniquely indicating a set of the radiation imaging apparatus and the wireless master unit to be wirelessly connected, and is preferably information generated, for example, based on the first information uniquely indicating the radiation imaging apparatus and the second information uniquely indicating the wireless master unit. In this example, “FPD1-AP1”, which is obtained by connecting the information “FPD1” indicating the radiation imaging apparatus 100 and the information “AP1” indicating the first wireless master unit 114 by a hyphen “-”, is defined as the third information. Note that the third information generation method is not limited to this, and encryption may be performed by using an exclusive OR of both character strings, and the generation algorithm thereof is not limited. Further, the first information “FPD1” uniquely indicating the radiation imaging apparatus 100 is information recognized by both the radiation imaging apparatus 100 and the first wireless master unit 114. Therefore, only the second information “AP1” that uniquely indicates the first wireless master unit 114 may be transmitted from the first wireless master unit 114, and the third information may be generated by the radiation imaging apparatus 100 together with the first information “FPD1” that indicates the radiation imaging apparatus 100. In this case, it is necessary to use the same generation algorithm in the radiation imaging apparatus 100 and the first wireless master unit 114.

In step S1407, the controller 204 determines whether or not a predetermined period of time has elapsed since the transmission of the first information. If the controller 204 determines that the predetermined time has not elapsed (NO), the process returns to step S1406. On the other hand, when controller 204 determines that the predetermined time has elapsed (YES), the process returns to step S1402.

In step S1408, the setting unit 207 sets the acquired third information to the controller 204 as an identifier for establishing a second wireless connection with the wireless master unit. In this example, the setting unit 207 sets the SSID of the controller 204 to “FPD1-AP1”.

Next, in step S1409, the controller 204 determines whether or not a second wireless connection with the wireless master unit has been established. If the controller 204 determines that the second wireless connection with the wireless master unit has not been established (NO), the process proceeds to step S1410, and if the controller 204 determines that the second wireless connection with the wireless master unit has been established (YES), the process proceeds to step S1411. In this example, as shown in the state 1603 of FIG. 6, the third information “FPD1-AP1” is set as the SSID in the access point 112, whereby the second wireless connection can be established. In this state, a wireless connection with the first wireless master unit 114 is established to enable mutual communication.

In step S1410, the controller 204 determines whether or not a predetermined period of time has elapsed since it determined that the third information has been received. If the controller 204 determines that the predetermined time has not elapsed (NO), the process returns to step S1409. On the other hand, if the controller 204 determines that the predetermined time has elapsed (YES), the process returns to step S1401 to initialize the SSID.

When the controller 204 determines in step S1409 that the second wireless connection with the wireless master unit has been established, thereafter, data communication and the like are performed between the radiation imaging apparatus and the wireless master unit through the second wireless connection based on the third information uniquely indicating the combination. For example, in a state where the second wireless connection is maintained, photographing using the radiation imaging apparatus 100 is performed by the control software on the console. At this time, a command for controlling the radiation imaging apparatus 100, data indicating the state of the radiation imaging apparatus 100, radiation image data acquired by photographing, and the like are transmitted and received by the second wireless connection.

Here, it is assumed that, after the necessary photographing is performed, for example, the radiation imaging apparatus 100 is taken out for use in another radiation imaging chamber. In this case, since the identifier with the wireless master unit corresponding to the radiation imaging chamber used so far remains, the radiation imaging apparatus 100 cannot wirelessly connect with the wireless master unit corresponding to another radiation imaging chamber. It is necessary to initialize the current identifier in order to enable wireless connection with a wireless master unit corresponding to another radiographic chamber.

For example, when the second wireless connection based on the third information is interrupted for a predetermined time or more, it can be considered that the radiation imaging apparatus 100 has been taken out to another radiation imaging chamber. Therefore, as shown in step S1411, the controller 204 determines whether or not the second wireless connection has been interrupted for a predetermined period of time or longer. If the controller 204 determines that the second wireless connection has been interrupted for a predetermined time or longer (YES), the process returns to step S1401 to initialize the SSID.

Further, for example, the user may be able to input the initialization of the SSID via an operation unit (not shown) of the radiation imaging apparatus 100, and as shown in step S1412, the controller 204 determines whether or not the initialization of the SSID has been input. If it is determined that the SSID has been initialized (YES), the controller 204 notifies the wireless master of the SSID initialization instruction in step S1413, and then returns to step S1401 to initialize the SSID.

Further, for example, it is also possible to allow a user to input the initialization of the SSID from a touch panel or the like of a console constituting the wireless master unit, and in the wireless master unit, when there is the initialization input of the SSID, the wireless slave unit may be notified of the initialization instruction of the SSID. In this case, as shown in step S1414, the controller 204 determines whether or not an SSID initialization instruction has been received from the wireless master unit. If the controller 204 determines that an SSID initialization instruction has been received from the wireless master unit (YES), the process returns to step S1401 to initialize the SSID.

Next, an operation related to wireless connection on the wireless master unit side shown in FIG. 9 as an example will be described. In the radiation imaging system, in the initial state 1601 shown in FIG. 10, the wireless connection is not established because the wireless connection is not set. In the radiation imaging system, communication settings are initialized in each of the radiation imaging apparatus 100 and the access point 112 immediately after the system is started upon power-on. In the wireless master unit 114, in step S1501, the setting unit 1306 sets an identifier for establishing a first wireless connection that does not specify a communication partner in the controller 1303. In this example, the setting unit 1306 sets the SSID that is the identifier of the controller 1303 to “FREE”. As a result, the access point 112 of the wireless master unit 114 can establish a wireless connection without specifying a communication partner as shown in the state 1601 of FIG. 6.

Next, in step S1502, the controller 1303 determines whether or not a first wireless connection with the radiation imaging apparatus 100 as a wireless slave unit has been established. The controller 1303 repeats the processing of step S1502 until it is determined that the first wireless connection with the radiation imaging apparatus 100 is established, and when it is determined that the first wireless connection with the radiation imaging apparatus 100 is established, the process proceeds to step S1503. In this example, in the radiation imaging apparatus 100, since the information “FREE” that does not specify the communication partner is set as the SSID, the first wireless connection can be established, and in this state, the wireless connection can be established to communicate with each other. Therefore, when the first wireless connection is established, the states of the access point 112 and the radiation imaging apparatus 100 in this example become the state 1602 shown in FIG. 10.

After the first wireless connection is established, at step S1503, the controller 1303 transmits an information request of the first information uniquely indicating the radiation imaging apparatus 100 itself to the radiation imaging apparatus 100 through the first wireless connection.

After transmitting the information request of the first information, in step S1504, the controller 1303 determines whether or not the first information from the radiation imaging apparatus 100 has been received through the first wireless connection. If the controller 1303 determines that the first information has not been received from the radiation imaging apparatus 100 (NO), the process proceeds to step S1505, and if the controller 1303 determines that the first information has been received from the radiation imaging apparatus 100 (YES), the process proceeds to step S1506.

In step S1505, the controller 1303 determines whether or not a predetermined period of time has elapsed since the information request of the first information was transmitted. If the controller 1303 determines that the predetermined time has not elapsed (NO), the process returns to step S1504. On the other hand, when the controller 1303 determines that the predetermined time has elapsed (YES), the process returns to step S1502.

In step S1506, the notification unit 1304 generates third information for use as an SSID in the second wireless connection, and transmits the third information to the radiation imaging apparatus 100 on the wireless slave unit side via the controller 1303. The notification unit 1304 generates the third information based on the first information received from the radiation imaging apparatus 100 and the second information stored in the storage unit A 1305. The notification unit 1304 outputs the generated third information to the setting unit 1306.

Next, in step S1507, the setting unit 1306 sets the third information generated in step S1506 to the controller 1303 as an identifier for establishing a second wireless connection with the radiation imaging apparatus 100. In this example, the setting unit 1306 sets the SSID of controller 1303 to “FPD1-AP1”.

Next, in step S1508, the controller 1303 determines whether or not a second wireless connection with the wireless slave unit has been established. If the controller 1303 determines that the second wireless connection with the wireless slave unit is not established (NO), the process proceeds to step S1509, and if the controller 1303 determines that the second wireless connection with the wireless slave unit is established (YES), the process proceeds to step S1510. In this example, when the third information “FPD1-AP1” is set as the SSID in the first wireless master unit 114, the second wireless connection can be established, and in this state, the wireless connection can be established to enable mutual communication.

In step S1509, the controller 1303 determines whether or not a predetermined period of time has elapsed since the SSID was set to the third information. If the controller 1303 determines that the predetermined time has not elapsed (NO), the process returns to step S1508. On the other hand, if controller 1303 determines that the predetermined time has elapsed (YES), the process returns to step S1501 to initialize the SSID.

In step S1510, the controller 1303 determines whether or not the wirelessly connected wireless slave unit is the radiation imaging apparatus 100 to be used for photographing. If it is determined that the wirelessly connected wireless slave unit is not the radiation imaging apparatus 100 to be used (NO), the process returns to step S1501 to initialize the SSID.

When the second wireless connection between the radiation imaging apparatus 100 and the first wireless master unit 114 is established, the states of the first access point 112 and the radiation imaging apparatus 100 in this example become the state 1603 shown in FIG. 10. In the state 1603, since the first access point 112 and the radiation imaging apparatus 100 implement unique wireless connection based on the SSID that uniquely indicates the combination of both, erroneous connection to other wireless networks is prevented.

When the controller 1303 determines that the second wireless connection with the radiation imaging apparatus 100 has been established, thereafter, data communication and the like are performed between the radiation imaging apparatus 100 and the wireless master unit 114 through the second wireless connection based on the third information uniquely indicating the combination. For example, in a state where the second wireless connection is maintained, photographing using the radiation imaging apparatus 100 is performed by the control software on the console, and various commands, various data, and the like are transmitted and received by the second wireless connection.

Here, it is assumed that after the necessary photographing is performed, for example, the radiation imaging apparatus 100 is taken out for use in another radiation imaging chamber. In this case, when another radiation imaging apparatus is brought in, wireless connection with the radiation imaging apparatus becomes impossible if the previous identifier remains. To allow wireless connection to another radiation imaging apparatus, the current identifier must be initialized.

For example, when the second wireless connection based on the third information is interrupted for a predetermined time or more, it can be considered that the radiation imaging apparatus 100 has been taken out to another radiation imaging chamber. Therefore, as shown in step S1511, the controller 1303 determines whether or not the second wireless connection has been interrupted for a predetermined period of time or longer. If the controller 1303 determines that the second wireless connection has been interrupted for a predetermined time or longer (YES), the process returns to step S1501 to initialize the SSID.

Further, for example, the user may be able to input the initialization of the SSID via the console, and as shown in step S1512, the controller 1303 determines whether or not the initialization of the SSID has been input. If it is determined that the SSID has been initialized (YES), the controller 1303 notifies the wireless slave unit side of the SSID initialization instruction in step S1513, and then returns to step S1501 to initialize the SSID.

Further, for example, it is also possible to enable a user to input the initialization of the SSID via an operation unit (not shown) of the radiation imaging apparatus, and in the radiation imaging apparatus, when there is the initialization input of the SSID, the initialization instruction of the SSID may be notified to the wireless master unit side. In this case, as shown in step S1514, the controller 1303 determines whether or not an SSID initialization instruction has been received from the wireless slave unit side. If the controller 1303 determines that an SSID initialization instruction has been received from the wireless slave unit side (YES), the process returns to step S1501 to initialize the SSID.

According to the second embodiment, information that does not specify a communication partner is set as an identifier to establish a first wireless connection between the radiation imaging apparatus 100 and the wireless master unit. A second wireless connection is established by wireless communication in the first wireless connection by third information uniquely indicating the combination of the radiation imaging apparatus 100 and the wireless master unit generated from first information uniquely indicating the radiation imaging apparatus 100 and second information uniquely indicating the wireless master unit. Thus, erroneous connection to another wireless network can be prevented without any special operation by the user, and wireless connection to a desired wireless network can be established.

### Third Embodiment

Next, a third embodiment of the present disclosure will be described. In the second embodiment, a case has been described in which a second wireless connection in which both apparatus and unit are specified unconditionally is established between a radiation imaging apparatus connected to an access point constituting a wireless master unit through a first wireless connection in which the other partner is not specified. In the third embodiment, a case will be described in which a second wireless connection is established between a radiation imaging apparatus selected from a list indicating a radiation imaging apparatus being connected based on information collected through a first wireless connection that does not specify a communication partner and a wireless master unit. In the following, only the difference from the second embodiment will be described in the third embodiment.

In the radiation imaging system according to the third embodiment, the unique information of the radiation imaging apparatus connected to the access point constituting the wireless master unit through the first wireless connection is collected, and a list indicating the radiation imaging apparatus being connected is generated. Then, a second wireless connection specifying both is established between the radiation imaging apparatus selected from the generated list and the wireless master unit.

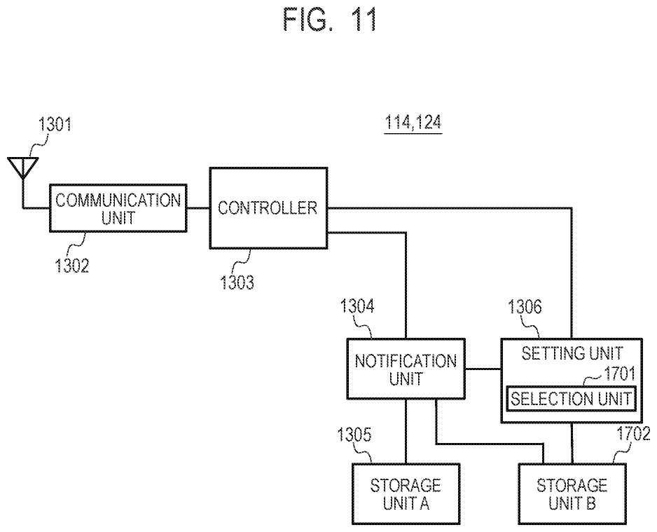

FIG. 11 is a block diagram showing a configuration example of the wireless master units 114 and 124 in the third embodiment. Each of the wireless master units 114 and 124 respectively includes a selection unit 1701 and a storage unit B 1702 in addition to a communication interface 1301, a communication unit 1302, a controller 1303, a notification unit 1304, a storage unit A 1305, and a setting unit 1306.

The selection unit 1701 selects information of a radiation imaging apparatus for establishing a second wireless connection from a list of information uniquely indicating the connected radiation imaging apparatus collected from the wirelessly connected radiation imaging apparatus and stored in the storage unit B 1702. The storage unit B 1702 stores various data including information related to wireless connection. The storage unit B 1702 stores first information which is collected through the first wireless connection and uniquely indicates each of all the radiation imaging apparatus connected to the wireless master units 114 and 124 through the first wireless connection in the communication network.

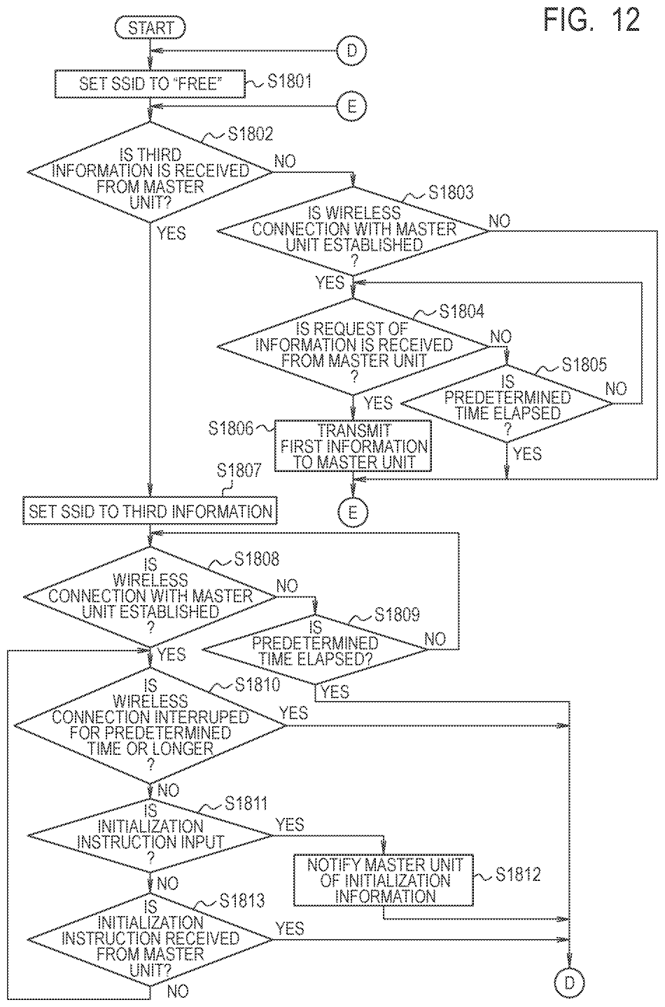

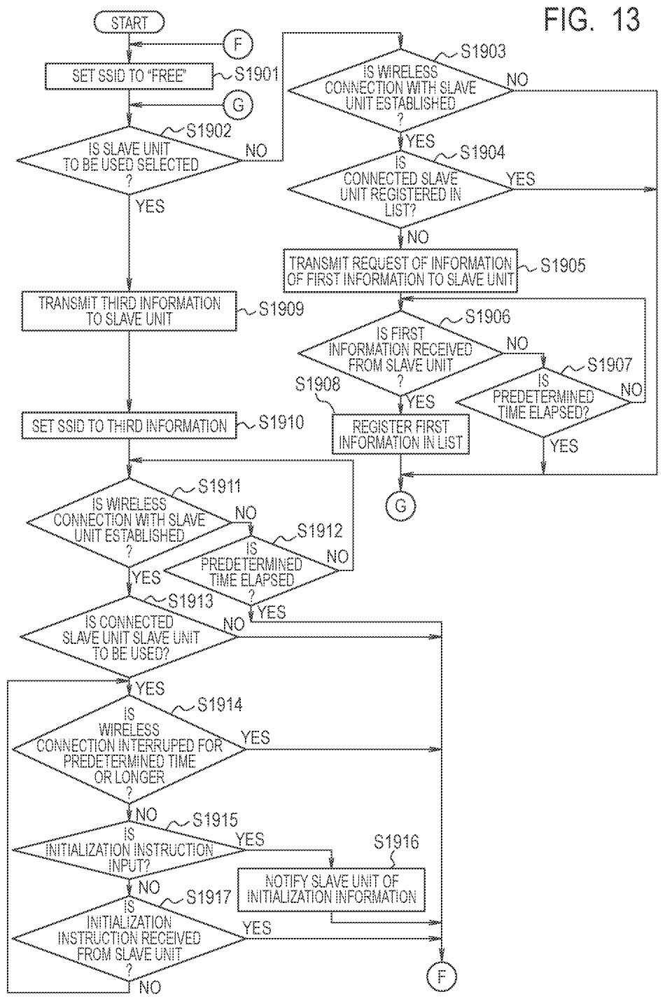

Next, referring to FIGS. 12 and 13, an operation related to wireless connection in the radiation imaging system of the third embodiment will be described. FIG. 12 is a flowchart showing an operation example related to wireless connection in the radiation imaging system (wireless slave unit side) in the third embodiment, and FIG. 13 is a flowchart showing an operation example related to wireless connection in the radiation imaging system (wireless master unit side) in the third embodiment. Hereinafter, a case where a wireless connection is established between the radiation imaging apparatus 100 and the first wireless master unit 114 constituted by the first access point 112 and the first console 113 will be described by way of example as appropriate.

First, an operation related to wireless connection in the radiation imaging apparatus 100, which is a wireless slave unit shown in FIG. 12, will be described. In the radiation imaging system, communication settings are initialized in each of the radiation imaging apparatus 100 and the access point 112 immediately after the system is started upon power-on. In the radiation imaging apparatus 100, in step S1801, the setting unit 207 sets an identifier for establishing a first wireless connection that does not specify a communication partner to the controller 204. In this example, the setting unit 207 sets the SSID that is the identifier of controller 204 to “FREE”. As a result, the radiation imaging apparatus 100 is in a state in which a first wireless connection that does not specify a communication partner can be established.

Next, in step S1802, the controller 204 determines whether or not the third information for use as the SSID in the second wireless connection has been received from the wireless master unit. In the second embodiment, when the radiation imaging apparatus 100 is selected on the wireless master unit side, the third information corresponding to the selected radiation imaging apparatus 100 is transmitted from the wireless master unit side. If the controller 204 determines that the third information has not been received from the wireless master unit (NO), the process proceeds to step S1803, and if the controller 204 determines that the third information has been received from the wireless master unit (YES), the process proceeds to step S1807.

Next, in step S1803, the controller 204 determines whether or not a first wireless connection with the wireless master unit has been established. If the controller 204 determines that the first wireless connection with the wireless base unit is established (YES), the process proceeds to step S1804, and if the controller 204 determines that the first wireless connection with the wireless base unit is not established (NO), the process returns to step S1802.

In step S1804 after the first wireless connection is established, the controller 204 determines whether or not the information request of the first information uniquely indicating the radiation imaging apparatus 100 itself has been received from the wireless master unit through the first wireless connection. If the controller 204 determines that the information request of the first information has not been received from the wireless master unit (NO), the process proceeds to step S1805, and if the controller 204 determines that the information request of the first information has been received from the wireless master unit (YES), the process proceeds to step S1806.

In step S1805, the controller 204 determines whether or not a predetermined period of time has elapsed since it determined that the first wireless connection has been established. If the controller 204 determines that the predetermined time has not elapsed (NO), the process returns to step S1804. On the other hand, if the controller 204 determines that the predetermined time has elapsed (YES), the process returns to step S1802.

In step S1806, the controller 204 acquires first information uniquely indicating the radiation imaging apparatus 100 itself, which is stored in the storage unit 208, and transmits the first information to the wireless master unit via the first wireless connection. After the processing in step S1806, the processing returns to step S1802.

In step S1802, if the controller 204 determines that the third information has been received, the processing from step S1807 onward proceeds similarly to the processing from step S1408 onward in the second embodiment shown in FIG. 8. That is, in step S1807, the setting unit 207 sets the acquired third information to the controller 204 as an identifier for establishing the second wireless connection with the wireless master unit. In this example, the setting unit 207 sets the SSID of the controller 204 to “FPD1-AP1”.

Next, in step S1808, the controller 204 determines whether or not a second wireless connection with the wireless master unit has been established. If the controller 204 determines that the second wireless connection with the wireless master unit has not been established (NO), the process proceeds to step S1809, and if the controller 204 determines that the second wireless connection with the wireless master unit has been established (YES), the process proceeds to step S1810.

In step S1809, the controller 204 determines whether or not a predetermined period of time has elapsed since it determined that the third information has been received. If the controller 204 determines that the predetermined time has not elapsed (NO), the process returns to step S1808, and if the controller 204 determines that the predetermined time has elapsed (YES), the process returns to step S1801.

In step S1808, when the controller 204 determines that the second wireless connection with the wireless master unit has been established, data communication or the like is subsequently performed between the radiation imaging apparatus and the wireless master unit through the second wireless connection. For example, in a state where the second wireless connection is maintained, photographing using the radiation imaging apparatus 100 is performed by the control software on the console.

Thereafter, similarly to step S1411 shown in FIG. 8, as shown in step S1810, the controller 204 determines whether or not the second wireless connection has been interrupted for a predetermined period of time or longer. If the controller 204 determines that the second wireless connection has been interrupted for a predetermined time or longer (YES), the process returns to step S1801.

Further, similarly to step S1412 shown in FIG. 8, as shown in step S1811, the controller 204 determines whether or not there is an initialization input of the SSID. If it is determined that the SSID has been initialized (YES), the controller 204 notifies the wireless master unit of the SSID initialization instruction in step S1812, and then returns to step S1801.

Similarly to step S1414 shown in FIG. 8, as shown in step S1813, the controller 204 determines whether or not an SSID initialization instruction has been received from the wireless master unit. If the controller 204 determines that an SSID initialization instruction has been received from the wireless master unit (YES), the process returns to step S1801 to initialize the SSID.

Next, an operation related to wireless connection on the wireless master unit side shown in FIG. 13 will be described. In the radiation imaging system, communication settings are initialized in each of the radiation imaging apparatus 100 and the access point 112 immediately after the system is started upon power-on. In the wireless master unit 114, in step 1901, the setting unit 1306 sets an identifier for establishing a first wireless connection that does not specify a communication partner in the controller 1303. In this example, the setting unit 1306 sets the SSID that is the identifier of controller 1303 to “FREE”. As a result, the access point 112 of the wireless master unit 114 can establish a first wireless connection without specifying a communication partner.

Next, in step S1902, the setting unit 1306 determines whether or not the radiation imaging apparatus 100 (wireless slave unit for establishing a second wireless connection) used for photographing at the console of the wireless master unit is selected. The radiation imaging apparatus is selected by, for example, displaying a list of radiation imaging apparatuses stored in the storage unit B 1702 on a screen of a console, and a user selects a desired radiation imaging apparatus by using a touch panel or the like. If the setting unit 1306 determines that the radiation imaging apparatus 100 to be used for photographing is not selected (NO), the processing proceeds to step S1903, and the processing of steps 1903 to 1908 is performed to perform the operation of collecting the first information of the radiation imaging apparatus connected by the first wireless connection. On the other hand, if the setting unit 1306 determines that the radiation imaging apparatus 100 to be used for photographing is selected (YES), the process proceeds to step S1909.

In step S1903, the controller 1303 determines whether or not a first wireless connection has been established with the radiation imaging apparatus 100 serving as a wireless slave unit. If the controller 1303 determines that the first wireless connection with the radiation imaging apparatus 100 has been established (YES), the process proceeds to step S1904. On the other hand, if the controller 204 determines that the first wireless connection with the radiation imaging apparatus 100 is not established (NO), the process returns to step S1902.

In step S1904, the controller 1303 determines whether or not the radiation imaging apparatus 100 connected by the first wireless connection is a radiation imaging apparatus registered in the list stored in the storage unit B 1702. If the controller 1303 determines that the radiation imaging apparatus is not registered in the list, that is, the radiation imaging apparatus is not registered (NO), the process proceeds to step S1905. On the other hand, if the controller 1303 determines that the radiation imaging apparatus is already registered in the list (YES), the process returns to step S1902.

In step S1905, the controller 1303 transmits an information request of the first information that uniquely identifies itself to the radiation imaging apparatus 100 to the radiation imaging apparatus 100 via the first wireless connection.

After transmitting the information request of the first information, in step S1906, the controller 1303 determines whether or not the first information from the radiation imaging apparatus 100 has been received through the first wireless connection. If the controller 1303 determines that the first information has not been received from the radiation imaging apparatus 100 (NO), the process proceeds to step S1907, and if controller 1303 determines that the first information has been received from the radiation imaging apparatus 100 (YES), the process proceeds to step S1908.

In step S1907, the controller 1303 determines whether or not a predetermined time has elapsed since the transmission of the information request of the first information. If the controller 1303 determines that the predetermined time has not elapsed (NO), the process returns to step S1906, and if the controller 1303 determines that the predetermined time has elapsed (YES), the process returns to step S1902.

In step S1908, the controller 1303 registers the first information received from the radiation imaging apparatus 100 in the list stored in the storage unit B 1702. After registering the first information of the radiation imaging apparatus 100 in the list stored in the storage unit B 1702, the process returns to step S1902. In the first wireless connection in which the communication partner is not specified, the first wireless master unit 114 repeats the processing of steps S1903 to S1908 each time a new radiation imaging apparatus is connected. Thus, the first wireless master unit 114 collects the first information from all the radiation imaging apparatuses connected to the access point 112, and creates a list of the radiation imaging apparatuses in the storage unit B 1702.

In step S1902, if the setting unit 1306 determines that the radiation imaging apparatus 100 to be used for photographing is selected, the processing from step S1909 onwards is the same as that from step S1506 onwards in the second embodiment shown in FIG. 9. That is, in step S1909, the notification unit 304 generates third information for use as the SSID in the second wireless connection, and transmits the third information to the radiation imaging apparatus 100 via the controller 1303. The notification unit 1304 outputs the generated third information to the setting unit 1306.

Next, in step S1910, the setting unit 1306 sets the third information generated in step S1909 to the controller 1303 as an identifier for establishing a second wireless connection with the radiation imaging apparatus 100. In this example, the setting unit 1306 sets the SSID of the controller 1303 to “FPD1-AP1”.

Next, in step S1911, the controller 1303 determines whether or not a second wireless connection with the wireless slave unit has been established. If the controller 1303 determines that the second wireless connection with the wireless slave unit is not established (NO), the process proceeds to step S1912, and if the controller 1303 determines that the second wireless connection with the wireless slave unit is established (YES), the process proceeds to step S1913.

In step S1912, the controller 1303 determines whether or not a predetermined period of time has elapsed since the SSID was set to the third information. If the controller 1303 determines that the predetermined time has not elapsed (NO), the process returns to step S1911, and if the controller 1303 determines that the predetermined time has elapsed (YES), the process returns to step S1901.

In step S1913, the controller 1303 determines whether or not the wirelessly connected wireless slave unit is the radiation imaging apparatus 100 to be used for photographing, that is, determines whether or not the radiation imaging apparatus 100 is selected in step S1902. If it is determined that the wirelessly connected wireless slave unit is not the radiation imaging apparatus 100 to be used (NO), the process returns to step S1901.

When the controller 1303 determines that the second wireless connection with the radiation imaging apparatus 100 has been established, data communication or the like is subsequently performed between the radiation imaging apparatus 100 and the wireless master unit 114 through the second wireless connection. For example, in a state where the second wireless connection is maintained, photographing using the radiation imaging apparatus 100 is performed by the control software on the console.

Thereafter, similarly to step S1511 shown in FIG. 9, as shown in step S1914, the controller 1303 determines whether or not the second wireless connection has been interrupted for a predetermined period of time or longer. If the controller 1303 determines that the second wireless connection has been interrupted for a predetermined time or longer (YES), the process returns to step S1901.

Similarly to step S1512 shown in FIG. 9, as shown in step S1915, the controller 1303 determines whether or not there is an initialization input of the SSID. If it is determined that the SSID has been initialized (YES), the controller 1303 notifies the wireless slave unit of the SSID initialization instruction in step S1916, and then returns to step S1901.

Similarly to step S1514 shown in FIG. 9, as shown in step S1917, the controller 1303 determines whether or not an SSID initialization instruction has been received from the wireless slave unit. If the controller 1303 determines that an SSID initialization instruction has been received from the wireless slave unit (YES), the process returns to step S1901.

According to the third embodiment, information that does not specify a communication partner is set as an identifier to establish a first wireless connection between the radiation imaging apparatus 100 and the wireless master unit. Then, first information uniquely indicating the radiation imaging apparatus 100 is collected by wireless communication in the first wireless connection, and a second wireless connection between the selected radiation imaging apparatus 100 and a wireless master unit is established by third information corresponding to the selected radiation imaging apparatus 100. Thus, a user can prevent erroneous connection to another wireless network without performing a special operation related to setting, and can establish wireless connection to a desired wireless network.

According to the present invention, in a radiation imaging system, erroneous connection to another wireless network can be prevented without any special operation by a user, and wireless connection to a desired wireless network can be established.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

It should be noted that the foregoing embodiments are merely illustrative examples of the embodiments of the present invention, and the technical scope of the present invention should not be interpreted in a limited manner. That is, the present invention is not limited to the embodiments described above, and various modifications and variations are possible without departing from the spirit and scope of the present invention. Accordingly, in order to make the scope of the present invention public, the following claims are attached.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A radiation imaging system for wirelessly connecting a control apparatus and a radiation imaging apparatus controlled by the control apparatus via any one of a plurality of access points, comprising:
a first storage unit included within the radiation imaging apparatus, and configured to store first information uniquely indicating the radiation imaging apparatus;
a second storage unit included within at least one of an access point of the plurality of access points and the control apparatus connected to the access point, and configured to store second information uniquely indicating the access point;
a third storage unit included within at least one of the access point and the control apparatus connected to the access point, and configured to store third information uniquely indicating a radiation imaging apparatus that can be wirelessly connected to the access point;
a first control unit included within the radiation imaging apparatus, configured to establish a first wireless connection with a first access point of the plurality of access points by using the first information as an identifier, and receive fourth information corresponding to the second information through the first wireless connection so as to establish a second wireless connection with the first access point by using the fourth information as the identifier; and
a second control unit included within the access point, configured to establish the first wireless connection with the radiation imaging apparatus using the third information corresponding to the selected radiation imaging apparatus as an identifier, so as to transmit the fourth information through the first wireless connection, and establish the second wireless connection with the radiation imaging apparatus using the fourth information as the identifier.

2. A radiation imaging system according to claim 1, wherein the fourth information is information uniquely indicating a combination of the radiation imaging apparatus and the first access point connected by the first wireless connection.

3. A radiation imaging system according to claim 1, wherein the fourth information is information generated based on the second information and the third information.

4. A radiation imaging system according to claim 1, wherein the identifier includes at least one of an SSID and an encryption key, associated with a wireless connection.

5. A radiation imaging system according to claim 1, wherein the access point includes selecting unit configured to select the third information corresponding to the selected radiation imaging apparatus, from the third storage unit.

6. A radiation imaging system according to claim 1, wherein the third information is the same as the first information of the corresponding radiation imaging apparatus.

7. A radiation imaging system according to claim 1, wherein the first information is set as the identifier in the radiation imaging apparatus when the second wireless connection between the radiation imaging apparatus and the first access point is interrupted for a predetermined time or longer, when an initialization instruction is received, or when a power supply is turned on.

8. A radiation imaging system according to claim 1, wherein the first access point initializes the identifier when the second wireless connection between the first access point and the radiation imaging apparatus is interrupted for a predetermined time or longer, when an initialization instruction is received, or when power is turned on.

9. A control method for controlling a radiation imaging system in which a control apparatus and a radiation imaging apparatus controlled by the control apparatus are wirelessly connected via any one of a plurality of access points, wherein
first information uniquely indicating the radiation imaging apparatus, is stored in a first storage unit included within the radiation imaging apparatus,
second information uniquely indicating the access point is stored in a second storage unit included within at least one of an access point of the plurality of access points and the control apparatus connected to the access point, and
third information uniquely indicating a radiation imaging apparatus which can be wirelessly connected to the access point, is stored in a third storage unit of at least one of the access point and the control apparatus connected to the access point,
the control method comprising the steps of:
establishing a first wireless connection between the radiation imaging apparatus and the first access point of the plurality of the access points, by using the first information stored in the first storage unit by the radiation imaging unit as an identifier, and by using third information corresponding to a selected radiation imaging apparatus among the third information stored in the third storage unit as an identifier; and
establishing a second wireless connection between the radiation imaging apparatus and the first access point by receiving fourth information corresponding to the second information through the first wireless connection so as to use the fourth information as an identifier, and by using the fourth information as an identifier by the first access point.

10. A radiation imaging system for wirelessly connecting a control apparatus and a radiation imaging apparatus controlled by the control apparatus via any one of a plurality of access points, comprising:
a first storage unit included within the radiation imaging apparatus, and configured to store first information uniquely indicating the radiation imaging apparatus;
a second storage unit included within at least one of an access point of the plurality of the access points and the control apparatus connected to the access point, and configured to store second information uniquely indicating the access point;
a first control unit included within the radiation imaging apparatus, and configured to establish a first wireless connection that does not specify a communication partner, transmit the first information through the first wireless connection, receive third information corresponding to the second information through the first wireless connection, and establish a second wireless connection with an access point of the plurality of access points by using the third information as an identifier; and
a second control unit included within the access point, and configured to establish the first wireless connection that does not specify a communication partner, receive the first information through the first wireless connection, transmit the third information to establish the second wireless connection with the radiation imaging apparatus using the third information as an identifier.

11. A radiation imaging system according to claim 10, wherein the third information is information uniquely indicating a combination of the radiation-imaging apparatus and the access point to be wirelessly connected.

12. A radiation imaging system according to claim 10, wherein the third information is information generated based on the first information and the second information.

13. A radiation imaging system according to claim 10, wherein the second control unit requests the first information from the radiation imaging apparatus connected by said first wireless connection.

14. A radiation imaging system according to claim 10, wherein the second control unit establishes the second wireless connection with the radiation imaging apparatus selected from among radiation imaging apparatus connected by the first wireless connection.

15. A radiation imaging system according to claim 10, wherein the identifier includes at least one of an SSID and an encryption key associated with a wireless connection.

16. A radiation imaging system according to claim 10, wherein the first control unit performs processing for establishing the first wireless connection when the second wireless connection between the radiation imaging apparatus and the access point is interrupted for a predetermined time or longer, when an initialization instruction is received, or when the radiation imaging apparatus is powered on.

17. A radiation imaging system according to claim 10, wherein the second control unit performs processing for establishing the first wireless connection when the second wireless connection between the access point and the radiation imaging apparatus is interrupted for a predetermined time or longer, when an initialization instruction is received, or when said access point is powered on.

18. A control method for controlling a radiation imaging system in which a control apparatus and a radiation imaging apparatus controlled by the control apparatus are wirelessly connected via any one of a plurality of access points,
the control method comprising the steps of:
establishing a first wireless connection that does not specify a communication partner, so that the radiation imaging apparatus transmits a first information uniquely indicating the radiation imaging apparatus through the first wireless connection;
causing the access point to receive the first information through the first wireless communication and to transmit to the radiation imaging apparatus a third information corresponding to the first information and a second information uniquely indicating the access point; and
establishing a second wireless connection between the radiation imaging apparatus and the access point of the plurality of access points by using the third information as an identifier.
